(19) United States
(12) Patent Application Publication
(10) Pub. No.: US 2024/0197750 A1
(43) Pub. Date: Jun. 20, 2024

(54) CEFTIBUTEN DOSING REGIMENS

(71) Applicant: QPEX Biopharma, Inc., San Diego, CA (US)

(72) Inventors: David C. Griffith, San Diego, CA (US); Olga Lomovskaya, San Diego, CA (US); Elizabeth E. Morgan, San Diego, CA (US); Jeffery S. Loutit, San Diego, CA (US)

(21) Appl. No.: 18/553,769

(22) PCT Filed: Mar. 31, 2022
(86) PCT No.: PCT/US2022/071479

§ 371 Date: Oct. 3, 2023

Related U.S. Application Data
(60) Provisional application No. 63/170,936, filed on Apr. 5, 2021.

(51) Int. Cl. A61K 31/546; A61K 31/69

(52) U.S. Cl. CPC A61K 31/546; A61K 31/69

(57) ABSTRACT

Disclosed herein are dosing regimens of ceftibuten in combination with a β-lactamase inhibitor. The dosing regimens include ceftibuten at a dose higher than 400 mg per day (for example, 400 mg BID, 600 mg QD or BID, or 800 mg QD or BID).

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims the benefit of U.S. provisional application No. 63/170,936, filed Apr. 5, 2021, which is incorporated in its entirety.

### STATEMENT REGARDING FEDERALLY SPONSORED R&D

This invention was made with U.S. government support under the Department of Health and Human Services Contract No. HHSO100201600026C. The U.S. government has certain rights in the invention.

### BACKGROUND OF THE INVENTION

### Field of the Invention

This application relates to the field of medicine, and in particular, ceftibuten and its formulations and use.

### Description of the Related Art

Ceftibuten is a third-generation cephalosporin antibiotic. It is approved by the FDA for oral administration at a dose of 400 mg once a day. Ceftibuten is susceptible to development of bacterial resistance. Accordingly, there is a need for new therapies using ceftibuten that can be effective against resistance development.

### SUMMARY OF THE INVENTION

Disclosed herein is pharmaceutical composition, comprising ceftibuten in an amount greater than 400 mg and a β-lactamase inhibitor. Also disclosed herein is a method of treating a bacterial infection, comprising co-administering to a subject in need thereof ceftibuten in an amount greater than 400 mg and a β-lactamase inhibitor.

### BRIEF DESCRIPTION OF THE DRAWINGS

FIG. 1 is a graph depicting AUC₀₋₁₂ of cis-ceftibuten after administration of 400 mg, 600 mg, or 800 mg doses.

FIG. 2 is a graph depicting AUC₀₋₁₂ of trans-ceftibuten after administration of 400 mg, 600 mg, or 800 mg doses.

### DETAILED DESCRIPTION OF THE PREFERRED EMBODIMENT

Some embodiments disclosed herein are directed to treating a bacterial infection by administering ceftibuten in combination with a β-lactamase inhibitor. In some embodiments, the dose of ceftibuten is higher than 400 mg. As described below, it was surprisingly discovered that administration of doses of ceftibuten higher than 400 mg results in dose-proportional exposure. In addition, it was discovered that higher concentrations of ceftibuten can decrease the development of bacterial resistance in combination with a β-lactamase inhibitor. Taken together, these discoveries support administration of ceftibuten at doses higher than 400 mg in combination with a β-lactamase inhibitor

In various embodiments, the dose of ceftibuten is higher than 450 mg, 500 mg, 550 mg, 600 mg, 750 mg, 800 mg, or 850 mg. In some embodiments, the dose of ceftibuten is in a range between any two doses selected from 400 mg, 450 mg, 500 mg, 550 mg, 600 mg, 750 mg, 800 mg, and 850 mg. In various embodiments, the dose of ceftibuten is administered once, twice, or three times a day. In some embodiments, 400 mg of ceftibuten is administered twice a day. In some embodiments, the ceftibuten and β-lactamase inhibitor are administered in the same formulation. In other embodiments, the ceftibuten and β-lactamase inhibitor are administered in separate formulations.

### Definitions

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkylthio” refers to the formula —SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only. “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

As used herein, “alkylene” means a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” means a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl.” “C₆ or C₁₀ aryl.” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such as “C₇₋₁₄ aralkyl” and the like, including but not limited to benzyl, 2-phenylethyl. 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is selected from hydrogen, optionally substituted C₁₋₆ alkyl, halogen, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, optionally substituted C₁₋₆ alkyl, halogen, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

A “C-carboxy” group refers to a “—C(═O)OR” group in which R is selected from hydrogen, halogen, optionally substituted C₁₋₆ alkyl, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, optionally substituted C₁₋₆ alkyl, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

A “sulfonyl” group refers to an “—SO₂R” group in which R is selected from hydrogen, optionally substituted C₁₋₆ alkyl, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

An “S-sulfonamido” group refers to a “—SO₂NR_{A}R_{B}” group in which R_{A} and R_{B} are each independently selected from hydrogen, halogen, optionally substituted C₁₋₆ alkyl, optionally substituted C₁₋₆ alkoxy, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

An “N-sulfonamido” group refers to a “—N(R_{A})SO₂R_{B}” group in which R_{A} and R_{B} are each independently selected from hydrogen, halogen, optionally substituted C₁₋₆ alkyl, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

A “C-amido” group refers to a “—C(═O)NR_{A}R_{B}” group in which R_{A} and R_{B} are each independently selected from hydrogen, halogen, optionally substituted C₁₋₆ alkyl, optionally substituted C₁₋₆ alkoxy, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

An “N-amido” group refers to a “—N(R_{A})C(═O)R_{B}” group in which R_{A} and R_{B} are each independently selected from hydrogen, halogen, optionally substituted C₁₋₆ alkyl, optionally substituted C₁₋₆ alkoxy, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, a C₆₋₁₀ ryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, halogen, optionally substituted C₁₋₆ alkyl, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

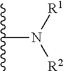

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

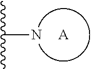

where ring A is a heteroaryl ring containing the depicted nitrogen.

Similarly, when two “adjacent” R groups are said to form a ring “together with the atom to which they are attached,” it is meant that the collective unit of the atoms, intervening bonds, and the two R groups are the recited ring. For example, when the following substructure is present:

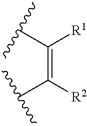

and R¹ and R² are defined as selected from the group consisting of hydrogen and alkyl, or R¹ and R² together with the atoms to which they are attached form an aryl or carbocylyl, it is meant that R¹ and R² can be selected from hydrogen or alkyl, or alternatively, the substructure has structure:

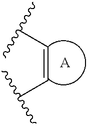

where A is an aryl ring or a carbocylyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

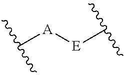

includes the substituent being oriented such that the A is attached at the leftmost attachment point of the molecule as well as the case in which A is attached at the rightmost attachment point of the molecule.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

“Solvate” refers to the compound formed by the interaction of a solvent and a compound described herein, a metabolite, or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

### β-Lactamase Inhibitors

In some embodiments, the β-lactamase inhibitor for use as described herein is a compound having the structure of any one of Formulas (I)-(IX):

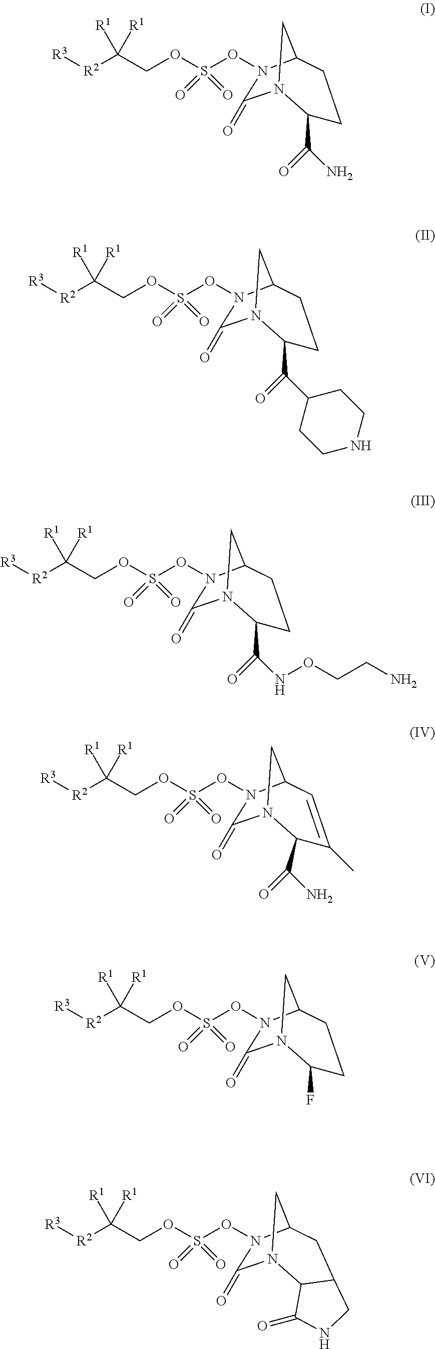

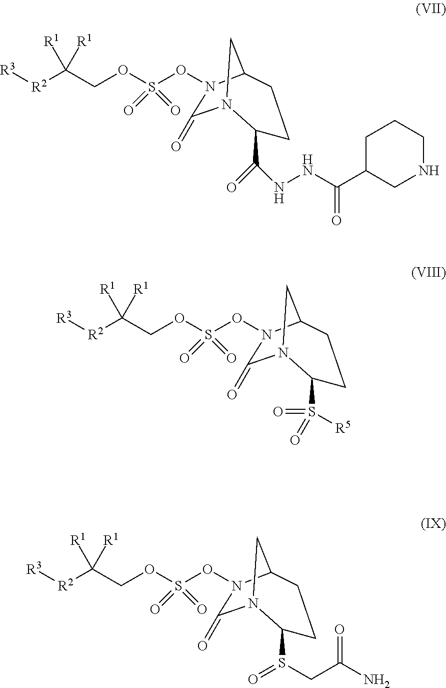

or pharmaceutically acceptable salts thereof, wherein:
each R¹ is independently a C₁₋₆ alkyl, or each R¹ together with the geminal carbon atom to which they are bonded forms an optionally substituted C₃₋₆ cycloalkyl ring or an optionally substituted 4-6 membered heterocycloalkyl ring;
R² is selected from a single bond, optionally substituted C₁₋₆ alkyl, optionally substituted 2-6 membered heteroalkyl, optionally substituted C₅₋₆ cycloalkyl, optionally substituted 5-6 membered heterocycloalkyl, optionally substituted phenyl, and optionally substituted 5-6 membered heteroaryl;
R³ is selected from C₁₋₆ alkyl, —O—C(O)—R⁴, —S—C(O)—R⁴, —NH—C(O)—R⁴, —O—C(O)—O—R⁴, —S(O)—O—R⁴, —NH—C(O)—O—R⁴, —(O)—O—R⁴, —C(O)—S—R⁴, —C(O)—NH—R⁴, —O—(O)—O—R⁴, —O—C(O)—S—R⁴, —O—C(O)—NH—R⁴, —S—S—R⁴, —S—R⁴, —NH—R⁴, and —CH(—NH₂)—R⁴);
R⁴ is selected from hydrogen, optionally substituted C₁₋₈ alkyl, optionally substituted 2-8 membered heteroalkyl, optionally substituted C₅₋₈ cycloalkyl, optionally substituted 5-8 membered heterocycloalkyl, optionally substituted C₅₋₁₀ cycloalkylalkyl, optionally substituted 5-8 membered heterocycloalkyl-C₁₋₃-alkyl, optionally substituted phenyl, optionally substituted 5-8 membered heteroaryl, optionally substituted C₇₋₁₀ arylalkyl, and optionally substituted 5-8 membered heteroaryl-C₁₋₃-alkyl;

R⁵ is selected from the group consisting of C₁₋₆ alkyl, —NR⁶R⁷, —CH₂C(O)NH₂, and

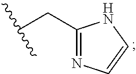 [SMILES: *Cc1ncc[nH]1]

and
R⁶ and R⁷ are independently selected from the group consisting of H, C₁₋₆ alkyl, and —CH₂C(O)NH₂.

Compounds of Formulas (I)-(IX) may be made following the procedures described in U.S. Pat. No. 10,085,999, which is incorporated herein by reference in its entirety.

In other embodiments, the β-lactamase inhibitor for use as described herein is a compound having the structure of any one of Formulas (X)-(XVII):

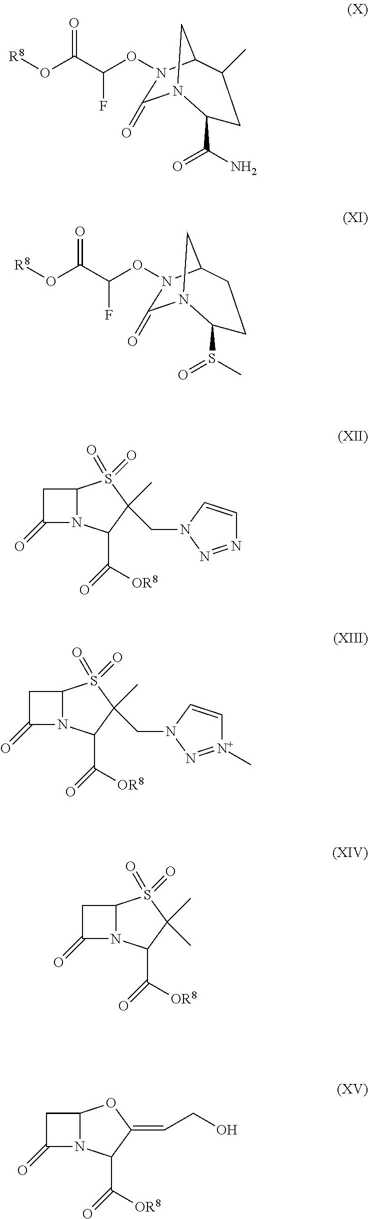

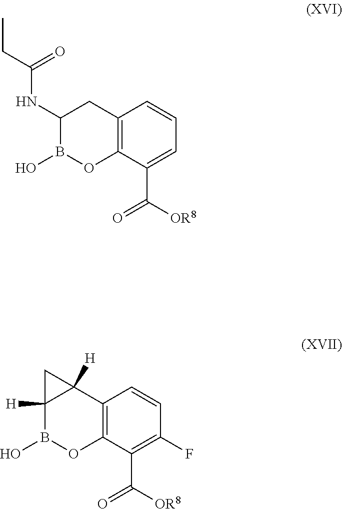

or pharmaceutically acceptable salts thereof, wherein:
R⁸ is selected from the group consisting of C₁₋₉ alkyl, —CR¹⁰R¹¹OC(O)C₁₋₉alkyl, —CR¹⁰R¹¹OC(O)C₃₋₇carbocyclyl, —CR¹⁰R¹¹OC(O)(3 to 7 membered heterocyclyl), —CR¹⁰R¹¹OC(O)C₂₋₈alkoxyalkyl, —CR¹⁰R¹¹OC(O)OC₁₋₉alkyl, —CR¹⁰R¹¹OC(O)OC₃₋₇carbocyclyl, —CR¹⁰R¹¹OC(O)O(3 to 7 membered heterocyclyl), —CR¹⁰R¹¹OC(O)OC₂₋₈alkoxyalkyl, —CR¹⁰R¹¹OC(O)C₆₋₁₀aryl, —CR¹⁰R¹¹OC(O)OC₆₋₁₀aryl, —CR¹⁰R¹¹C(O)NR¹³R¹⁴, —CR¹⁰R¹¹OC(O)O(CH₂)₁₋₃C(O)NR¹³R¹⁴, —CR¹⁰R¹¹OC(O)O(CH₂)₂₋₃OC(O)C₁₋₄alkyl, —CR¹⁰R¹¹OC(O)O(CH₂)₁₋₃C(O)OC₁₋₄ alkyl, —CR¹⁰R¹¹OC(O)(CH₂)₁₋₃OC(O)C₁₋₄ alkyl, and

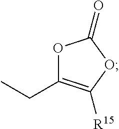

each R¹⁰ and R¹¹ is independently selected from the group consisting of H, optionally substituted C₁₋₄alkyl, optionally substituted C₃₋₇ carbocyclyl, optionally substituted 3-10 membered heterocyclyl, optionally substituted C₆₋₁₀aryl, and optionally substituted 5-10 membered heteroaryl;
each R¹³ and R¹⁴ is independently selected from the group consisting of H, optionally substituted C₁₋₆alkyl, optionally substituted C₃₋₇ carbocyclyl, optionally substituted 3-10 membered heterocyclyl, optionally substituted C₆₋₁₀aryl, and optionally substituted 5-10 membered heteroaryl; and
R¹⁵ is optionally substituted C₁₋₆ alkyl.

Compounds of Formulas (X)-(XVII) may be made using the procedures described in PCT Publication Nos. WO 2019/093450 or WO 2018/005662 (both of which are incorporated herein by reference in their entirety), or any other procedures for forming esters known in the art.

In some embodiments, the β-lactamase inhibitor for use as described herein is selected from the group consisting of:

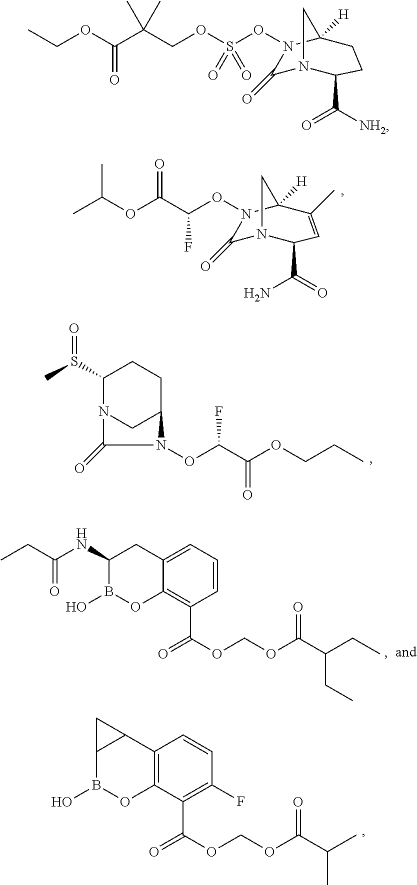

or pharmaceutically acceptable salts thereof. These compounds and their synthesis are described in U.S. Pat. No. 10,085,999, U.S. Application Publication No. 2019/0202832, PCT Publication No. WO2019/093450, PCT Publication No. WO2018/005662, and PCT Application No. PCT/US2021/022799, all of which are incorporated herein by reference in their entirety.

### Administration and Pharmaceutical Compositions

Administration of the compounds disclosed herein can be via any of the accepted modes of administration including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds disclosed can be formulated into pharmaceutical compositions, either individually or together. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) one or more compounds disclosed herein; and (b) a pharmaceutically acceptable carrier, diluent, excipient or combination thereof.

In addition to the selected compound useful as described above, some embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropics, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one or more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

Ceftibuten and the β-lactamase inhibitor may be co-administered. By “co-administered.” it is meant that the two agents are administered so as to have a biological effect at the same time, regardless of when or how they are actually administered. In some embodiments, the two agents may be found in the patient's bloodstream at the same time. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered intravenous (i.v.).

### Methods of Treatment

Some embodiments of the present invention include methods of treating bacterial infections with the compounds and compositions comprising the compounds described herein. Some methods include administering a compound, composition, pharmaceutical composition described herein to a subject in need thereof. In some embodiments, a subject can be an animal, e.g., a mammal (including a human). In some embodiments, the bacterial infection comprises a bacteria described herein. As will be appreciated from the foregoing, methods of treating a bacterial infection include methods for preventing bacterial infection in a subject at risk thereof.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

Bacterial infections that can be treated with the compounds, compositions and methods described herein can comprise a wide spectrum of bacteria. Example organisms include gram-positive bacteria, gram-negative bacteria, aerobic and anaerobic bacteria, such as Staphylococcus, Lactobacillus, Streptococcus, Sarcina, Escherichia, Enterobacter, Klebsiella, Pseudomonas, Acinetobacter, Mycobacterium, Proteus, Campylobacter, Citrobacter, Nisseria, Baccillus, Bacteroides, Peptococcus, Clostridium, Salmonella, Shigella, Serratia, Haemophilus, Brucella and other organisms.

More examples of bacterial infections include Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples. The following examples will further describe the present invention, and are used for the purposes of illustration only, and should not be considered as limiting.

### EXAMPLES

### Example 1

A double-blind, randomized, placebo-controlled, sequential ascending single- and multiple dose study of ceftibuten in healthy adult subjects was conducted. There were four cohorts of 10 subjects each who received both single and multiple oral doses (8 active and 2 placebo). The dosing scheme for Cohorts 1 to 4 is shown in Table 1.

TABLE 1
Cohorts 1 to 4 Dosing Scheme

| | | | Safety and PK |
|---|---|---|---|
| | Single-dose | Multiple-dose (BID) | Follow-up |
| Cohort | Day 1 | Days 4-10 | Days 11-13 |
| 1 | 8 subjects: ceftibuten | 8 subjects: ceftibuten | All subjects |
| | 400 mg QD | 400 mg BID | |
| | 2 subjects: placebo | 2 subjects: placebo | |
| 2 | 8 subjects: ceftibuten | 8 subjects: ceftibuten | All subjects |
| | 600 mg QD | 600 mg BID | |
| | 2 subjects: placebo | 2 subjects: placebo | |
| 3 | 8 subjects: ceftibuten | 8 subjects: ceftibuten | All subjects |
| | 800 mg QD | 800 mg BID | |
| | 2 subjects: placebo | 2 subjects: placebo | |
| 4 | 8 subjects: ceftibuten | 8 subjects: ceftibuten | All subjects |
| | 800 mg QD | 800 mg BID | |
| | 2 subjects: placebo | 2 subjects: placebo | |

### Safety

Ceftibuten in healthy volunteers was well tolerated in capsule formulation at 400 mg, 600 mg, and 800 mg QD/BID doses. The safety conclusions from the 40 enrolled subjects who received single and multiple doses of either ceftibuten or placebo over the duration of the study are as follows:
There were no serious, severe, life-threatening TEAEs, or TEAEs resulting in death reported throughout the study (Single ascending dose [SAD] and Multiple ascending dose [MAD] periods).
All TEAEs were mild, regardless of causality, with the exception of 2 TEAEs of nausea (SAD 800 mg ceftibuten) and vulvovaginal candidiasis (MAD 800 mg ceftibuten) which were moderate in severity. The event of nausea occurred in one subject in the SAD period following single administration of ceftibuten and the event of vulvovaginal candidiasis occurred in one subject in the MAD period following administration of the ceftibuten. Both events occurred in subjects receiving the highest dose of ceftibuten (800 mg) and were deemed by the investigator to be related to the study drug.
There were 2 TEAEs (nausea and diarrhea) in 1 subject from the highest dose group (800 mg ceftibuten) that led to the withdrawal of study drug, and the events of nausea also resulted in the subject being withdrawn from the study at the decision of the investigator. The nausea was considered to be moderate in severity and diarrhea considered to be mild in severity with both events deemed by the investigator to be related to the study drug. The nausea persisted from Day 1 dose (SAD) until after Day 4 morning dose (MAD), despite treatment with concomitant medications. The nausea resolved on Day 9 after the subject discontinued from the study.
In the SAD period of the study the frequency of reported TEAEs did not increase with increasing ceftibuten dose (Total Ceftibuten group had 44 TEAEs reported in 18 [56.3%] of 32 subjects, compared to the Pooled Placebo group with 5 TEAEs reported in 4 [50.0%] of 8 subjects).
Fifteen (15) treatment-related TEAEs were reported in 10 (25.0%) of 40 subjects during the SAD period of the study. The incidence of treatment-related TEAEs were similar for 400 mg and 600 mg ceftibuten, and Pooled Placebo groups (1 [12.5%] of 8 subjects) but was higher with the administration of 800 mg ceftibuten (4 [25.0%] of 16 subjects).
In the MAD period of the study the frequency of reported TEAEs did not increase with increasing ceftibuten dose, however the number of subjects reporting TEAEs in the Total Ceftibuten group was approximately twice that of the number of subjects reporting TEAEs in the Pooled Placebo group (22 [68.8%] of 32 subjects and 3 [37.5%] of 8 subjects, respectively).
In the MAD period of the study, 32 treatment-related TEAEs were reported in 17 (42.5%) of 40 subjects. The incidence of treatment-related TEAEs was similar in the 400 mg (1 [12.5%] of 8 subjects) and 600 mg (2 [25.0%] of 8 subjects) ceftibuten and Pooled Placebo groups (3 [37.5%] of 8 subjects). Treatment-related TEAEs were higher in the 800 mg ceftibuten group (11 [68.8%] of 16 subjects).
Treatment-related TEAEs were reported at the highest incidence in the SOC Gastrointestinal Disorders in the SAD period, MAD period and Combined SAD/MAD study. The highest incidence was reported in the highest ceftibuten dose (800 mg). The most common gastrointestinal TEAEs in the 800 mg group during the MAD period were nausea in 4 out of 16 subjects (25.0%), diarrhea in 4 of 16 (25.0%), abdominal pain in 3 of 16 (18.8%), upper abdominal pain in 3 of 16 (18.8%) and abdominal discomfort in 2 of 16 subjects (12.5%). Most Gastrointestinal Disorder TEAEs were transient and mild in severity with no intervention required. The exception was 1 treatment-related event of nausea which was moderate in severity reported in Subject 111-408 (800 mg ceftibuten).
ALT elevations occurred in some subjects across all ceftibuten dose groups in both the SAD and MAD periods of the study, the incidence and severity of which did not increase with increasing ceftibuten dose. All events were asymptomatic, resolved spontaneously after the completion of study treatment, and were not associated with elevations of alkaline phosphatase or bilirubin.
There was no trend in abnormalities that were NCS in hematology, coagulation, or urinalysis parameters. There were 4 events of abnormal findings in hematology, coagulation, or urinalysis that were considered to be clinically significant (CS): 3 events of low neutrophil counts and 1 event of low lymphocyte count, which were all asymptomatic and did not meet the criteria of an AE.
There were no CS abnormalities reported in ECG parameters or physical examination.
There were no trends in changes from Baseline in vital sign parameters of blood pressure (systolic and diastolic), pulse rate, body temperature or respiratory rate.

Overall, ceftibuten in healthy volunteers was well tolerated in capsule formulation at 400 mg, 600 mg, and 800 mg QD/BID doses.

### Pharmacokinetics

Following single doses of 400, 600 and 800 mg of ceftibuten, mean maximum cis-ceftibuten plasma concentrations reached 17.6, 24.1 and 28.1 μg/mL, respectively, and mean maximum trans-ceftibuten plasma concentrations reached 1.1, 1.5 and 2.2 μg/mL, respectively. Cis-ceftibuten and trans-ceftibuten pharmacokinetic parameters on Day 4 were similar to those on Day 1.

Following multiple doses of 400, 600 and 800 mg of ceftibuten, mean maximum cis-ceftibuten plasma concentrations reached 21.7, 28.1 and 38.8 μg/mL, respectively, and mean maximum trans-ceftibuten plasma concentrations reached 1.4, 1.9 and 2.8 μg/mL, respectively.

Cis-ceftibuten plasma concentrations peaked at 2.3-3.0 h post-dose, and trans-ceftibuten plasma concentrations peaked at 3.3-4.8 h post-dose. The terminal half-life of cis-ceftibuten ranged from 2.3-3.1 h, while the terminal-half-life of trans-ceftibuten ranged from 3.0-3.6 h. Total body clearance of cis-ceftibuten was in between 3.9-5.0 L/h.

After single doses, the urinary recovery as cis-ceftibuten and trans-ceftibuten accounted for 59.5-86.9% and 9.3-12.5% of the administered dose, respectively. After multiple doses, urinary recovery as cis-ceftibuten and trans-ceftibuten accounted for 75.8-100% and 14.1-16.8% of the administered dose, respectively. The renal clearance of cis-ceftibuten was estimated to be between 2.9-4.2 L/h. In general, renal clearance contributed a greater portion to the total clearance of ceftibuten.

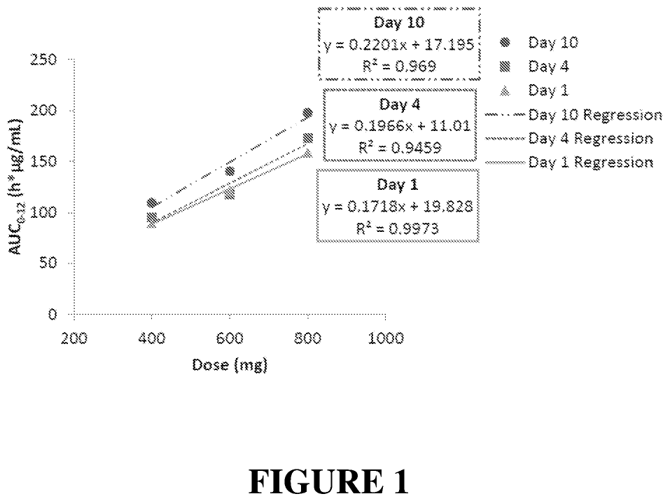

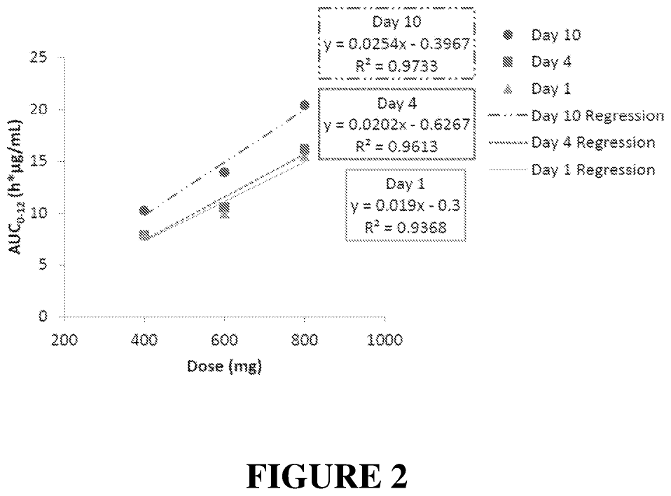

Dose proportionality was examined graphically and using an ANOVA test of the dose-normalized Cmax, AUC₀₋₁₂ and AUC_{0-∞} values. Cis-ceftibuten and trans-ceftibuten exposure increased proportionally following doses of 400, 600 and 800 mg of ceftibuten. The AUC₀₋₁₂ exposure of cis-ceftibuten is shown in FIG. 1 and the AUC₀₋₁₂ exposure of trans-ceftibuten is shown in FIG. 2, showing that increases were dose proportional.

### Example 2

99 strains of Enterobacterales were used in resistance development studies using ceftibuten at 1 or 2 μg/ml in combination with various concentrations of the following β-lactamase inhibitor:

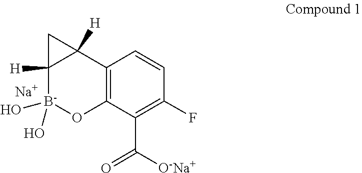

Compound 1 is described in PCT Publication No. WO 2018/005662, which is incorporated herein by reference in its entirety. Concentrations of Compound 1 included 1, 2, 4 and 8 μg/ml. The goal of the study was to identify the concentration of Compound 1 (MPC, mutant prevention concentration) that will prevent appearance of mutants at two different concentrations of ceftibuten. The data for Compound 1 at 4 μg/ml is summarized in Table 2.

TABLE 2
Bacterial resistance to ceftibuten + Compound 1 at 4 ug/ml.

| | | % Strains with | % Strains with |
|---|---|---|---|
| | | Compound 1 | Compound 1 |
| Ceftibuten/ | | MPC <= 4 ug/ml | MPC <= 4 ug/ml |
| Compound 1 | Number of | Ceftibuten at 1 | Ceftibuten at 2 |
| MIC | Strains at MIC | μg/ml | μg/ml |
| <=0.06 | 30 | 96.7 | 100.0 |
| 0.125 | 20 | 90.0 | 95.0 |
| 0.25 | 16 | 56.3 | 87.5 |
| 0.5 | 14 | 21.4 | 57.1 |
| 1 | 12 | 8.3 | 16.7 |
| 2 | 7 | 28.6 | 28.6 |

This data demonstrates that increasing the concentration of ceftibuten results in a higher percentage of strains for which resistance is prevented with Compound 1 at <=4 μg/ml. Thus, increasing the dose of ceftibuten above 400 mg in combination with a β-lactamase inhibitor can results in decreased development of resistance.

1. A pharmaceutical composition, comprising:
ceftibuten in an amount greater than 400 mg; and
a β-lactamase inhibitor.

2. The pharmaceutical composition of claim 1, wherein ceftibuten is present in an amount greater than 500 mg.

3. The pharmaceutical composition of claim 1, wherein ceftibuten is present in an amount greater than 550 mg.

4. The pharmaceutical composition of claim 1, wherein ceftibuten is present in an amount greater than 600 mg.

5. The pharmaceutical composition of claim 1, wherein ceftibuten is present in an amount greater than 650 mg.

6. The pharmaceutical composition of claim 1, wherein ceftibuten is present in an amount greater than 700 mg.

7. The pharmaceutical composition of claim 1, wherein ceftibuten is present in an amount greater than 750 mg.

8. The pharmaceutical composition of claim 1, wherein ceftibuten is present in an amount of about 600 mg.

9. The pharmaceutical composition of claim 1, wherein ceftibuten is present in an amount of about 800 mg.

10. The pharmaceutical composition of claim 1, wherein the β-lactamase inhibitor is a compound having the structure of any one of Formulas (I)-(IX): 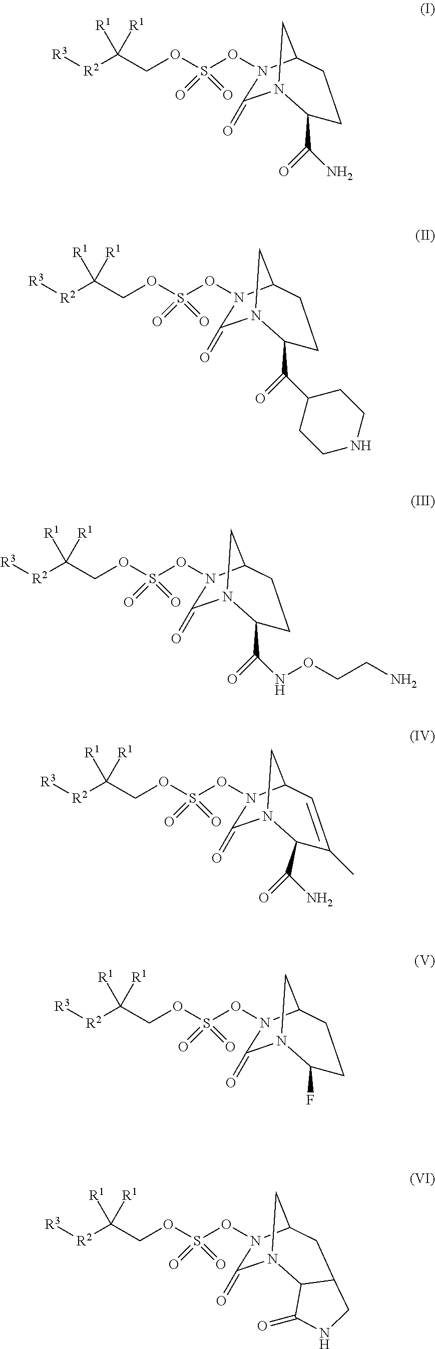 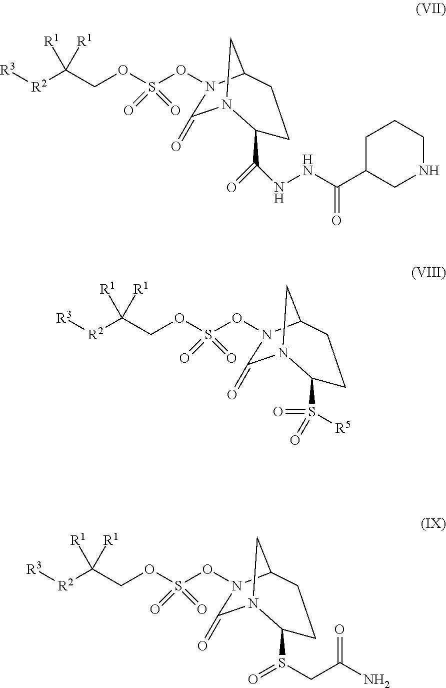
or pharmaceutically acceptable salts thereof, wherein:
each R¹ is independently a C₁₋₆ alkyl, or each R¹ together with the geminal carbon atom to which they are bonded forms an optionally substituted C₃₋₆ cycloalkyl ring or an optionally substituted 4-6 membered heterocycloalkyl ring;
R² is selected from a single bond, optionally substituted C₁₋₆ alkyl, optionally substituted 2-6 membered heteroalkyl, optionally substituted C₅₋₆ cycloalkyl, optionally substituted 5-6 membered heterocycloalkyl, optionally substituted phenyl, and optionally substituted 5-6 membered heteroaryl;
R³ is selected from C₁₋₆ alkyl, —O—C(O)—R⁴, —S—C(O)—R⁴, —NH—C(O)—R⁴, —O—C(O)—O—R⁴, —S(O)—O—R⁴, —NH—C(O)—O—R⁴, —(O)—O—R⁴, —C(O)—S—R⁴, —C(O)—NH—R⁴, —O—(O)—O—R⁴, —O—C(O)—S—R⁴, —O—C(O)—NH—R⁴, —S—S—R⁴, —S—R⁴, —NH—R⁴, and —CH(—NH₂)—R⁴);
R⁴ is selected from hydrogen, optionally substituted C₁₋₈ alkyl, optionally substituted 2-8 membered heteroalkyl, optionally substituted C₅₋₈ cycloalkyl, optionally substituted 5-8 membered heterocycloalkyl, optionally substituted C₅₋₁₀ cycloalkylalkyl, optionally substituted 5-8 membered heterocycloalkyl-C₁₋₃-alkyl, optionally substituted phenyl, optionally substituted 5-8 membered heteroaryl, optionally substituted C₇₋₁₀ arylalkyl, and optionally substituted 5-8 membered heteroaryl-C₁₋₃-alkyl;
R⁵ is selected from the group consisting of C₁₋₆ alkyl, —NR⁶R⁷, —CH₂C(O)NH₂, and 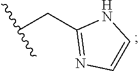 [SMILES: *Cc1ncc[nH]1]
and
R⁶ and R⁷ are independently selected from the group consisting of H, C₁₋₆ alkyl, and —CH₂C(O)NH₂.

11. The pharmaceutical composition of claim 1, wherein the β-lactamase inhibitor is a compound having the structure of any one of Formulas (X)-(XVII): 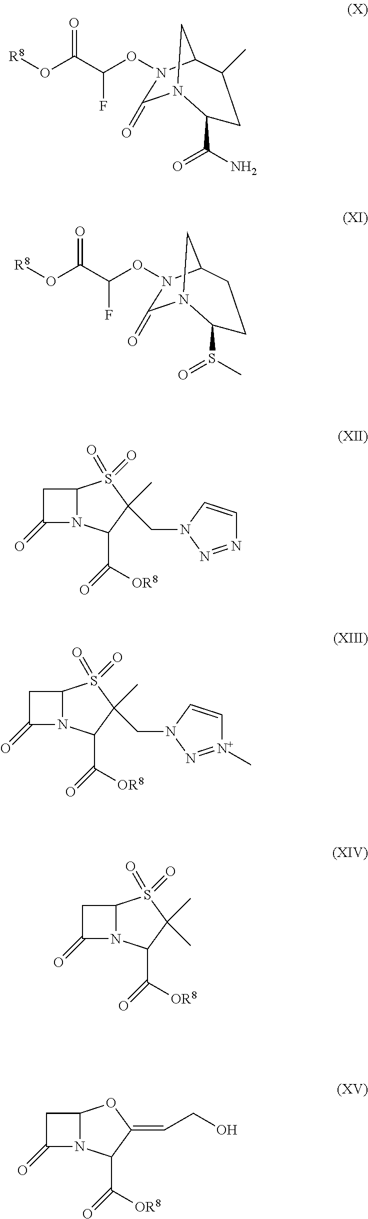 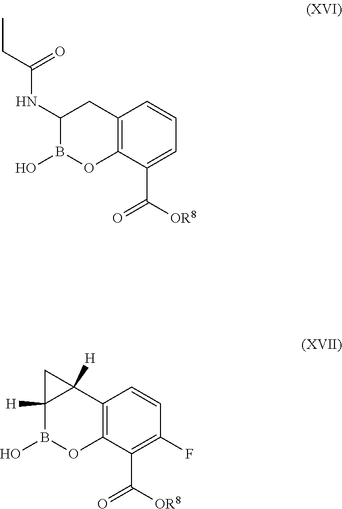
or pharmaceutically acceptable salts thereof, wherein:
R⁸ is selected from the group consisting of C₁₋₉ alkyl, —CR¹⁰R¹¹OC(O)C₁₋₉alkyl, —CR¹⁰R¹¹OC(O)C₃₋₇carbocyclyl, —CR¹⁰R¹¹OC(O)(3 to 7 membered heterocyclyl), —CR¹⁰R¹¹OC(O)C₂₋₈alkoxyalkyl, —CR¹OR¹¹OC(O)OC₁₋₉alkyl, —CR¹⁰R¹¹OC(O)OC₃₋₇carbocyclyl, —CR¹⁰R¹¹OC(O)O(3 to 7 membered heterocyclyl), —CR¹⁰R¹¹OC(O)OC₂₋₈alkoxyalkyl, —CR¹⁰R¹¹OC(O)C₆₋₁₀aryl, —CR¹⁰R¹¹OC(O)OC₆₋₁₀aryl, —CR¹⁰R¹¹C(O)NR¹³R¹⁴, —CR¹⁰R¹¹OC(O)O(CH₂)₁₋₃C(O)NR¹³R¹⁴, —CR¹⁰R¹¹OC(O)O(CH₂)₂₋₃OC(O)C₁₋₄ alkyl, —CR¹⁰R¹¹OC(O)O(CH₂)₁₋₃C(O)OC₁₋₄ alkyl, —CR¹⁰R¹¹OC(O)(CH₂)₁₋₃OC(O)C₁₋₄ alkyl, and 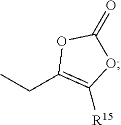
each R¹⁰ and R¹¹ is independently selected from the group consisting of H, optionally substituted C₁₋₄alkyl, optionally substituted C₃₋₇ carbocyclyl, optionally substituted 3-10 membered heterocyclyl, optionally substituted C₆₋₁₀aryl, and optionally substituted 5-10 membered heteroaryl;
each R¹³ and R¹⁴ is independently selected from the group consisting of H, optionally substituted C₁₋₆alkyl, optionally substituted C₃₋₇ carbocyclyl, optionally substituted 3-10 membered heterocyclyl, optionally substituted C₆₋₁₀aryl, and optionally substituted 5-10 membered heteroaryl; and
R¹⁵ is optionally substituted C₁₋₆ alkyl.

12. The pharmaceutical composition of claim 1, wherein the β-lactamase inhibitor is selected from the group consisting of: 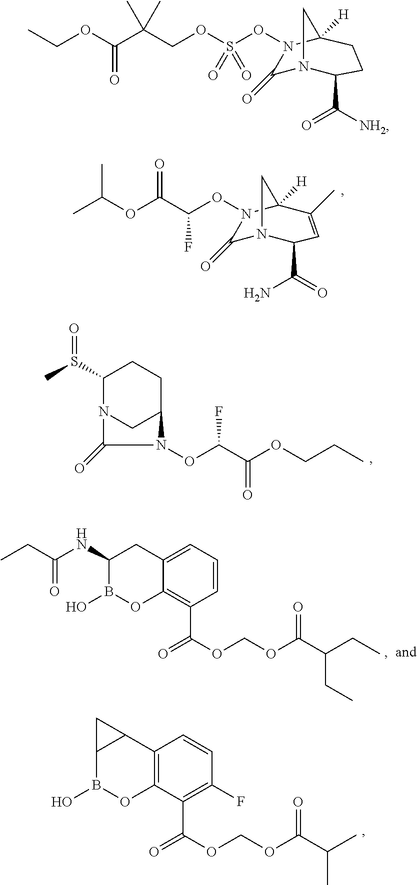
or pharmaceutically acceptable salts thereof.

13. A method of treating a bacterial infection, comprising co-administering to a subject in need thereof ceftibuten in an amount greater than 400 mg and a β-lactamase inhibitor.

14. The method of claim 13, wherein the ceftibuten is administered in an amount greater than 500 mg.

15. The method of claim 13, wherein the ceftibuten is administered in an amount greater than 550 mg.

16. The method of claim 13, wherein the ceftibuten is administered in an amount greater than 600 mg.

17. The method of claim 13, wherein the ceftibuten is administered in an amount greater than 650 mg.

18. The method of claim 13, wherein the ceftibuten is administered in an amount greater than 700 mg.

19. The method of claim 13, wherein the ceftibuten is administered in an amount greater than 750 mg.

20. The method of claim 13, wherein the ceftibuten is administered in an amount of about 600 mg.

21. The method of claim 13, wherein the ceftibuten is administered in an amount of about 800 mg.

22. The method of claim 13, wherein the β-lactamase inhibitor is a compound having the structure of any one of Formulas (I)-(IX): 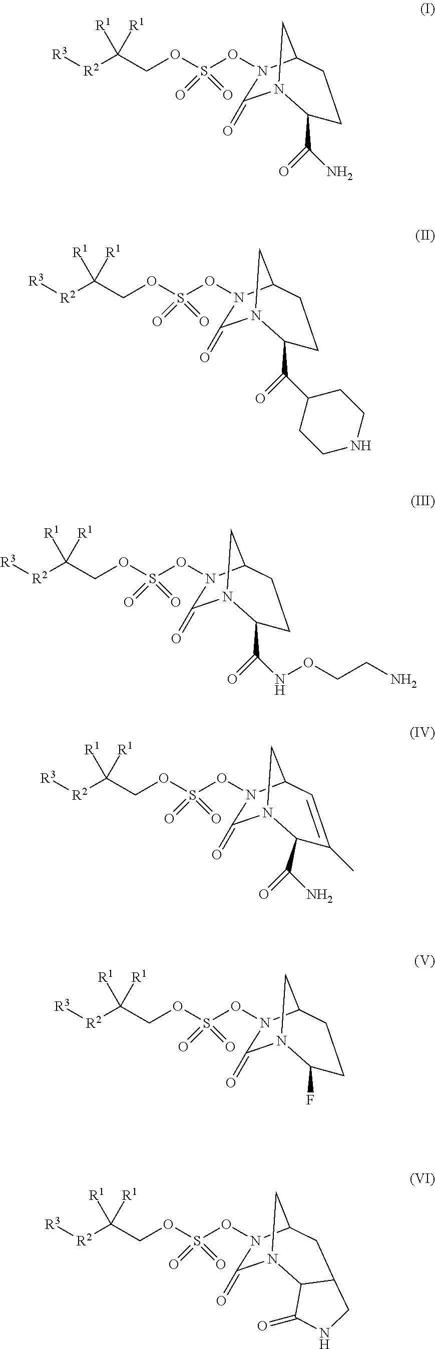 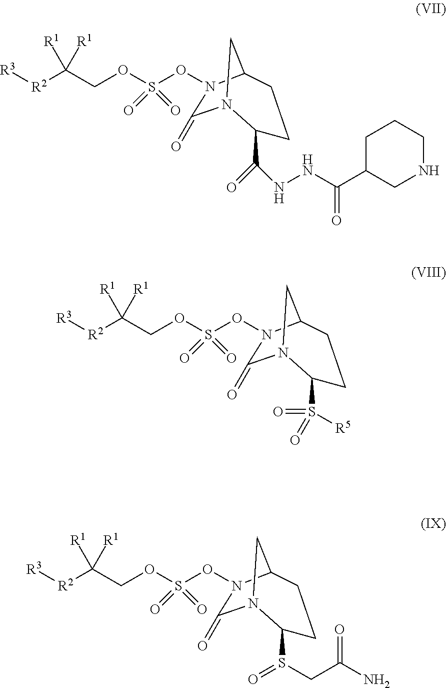
or pharmaceutically acceptable salts thereof, wherein:
each R¹ is independently a C₁₋₆ alkyl, or each R¹ together with the geminal carbon atom to which they are bonded forms an optionally substituted C₃₋₆ cycloalkyl ring or an optionally substituted 4-6 membered heterocycloalkyl ring;
R² is selected from a single bond, optionally substituted C₁₋₆ alkyl, optionally substituted 2-6 membered heteroalkyl, optionally substituted C₅₋₆ cycloalkyl, optionally substituted 5-6 membered heterocycloalkyl, optionally substituted phenyl, and optionally substituted 5-6 membered heteroaryl;
R³ is selected from C₁₋₆ alkyl, —O—C(O)—R⁴, —S—C(O)—R⁴, —NH—C(O)—R⁴, —O—C(O)—O—R⁴, —S(O)—O—R⁴, —NH—C(O)—O—R⁴, —(O)—O—R⁴, —C(O)—S—R⁴, —C(O)—NH—R⁴, —O—(O)—O—R⁴, —O—C(O)—S—R⁴, —O—C(O)—NH—R⁴, —S—S—R⁴, —S—R⁴, —NH—R⁴, and —CH(—NH₂)—R⁴);
R⁴ is selected from hydrogen, optionally substituted C₁₋₈ alkyl, optionally substituted 2-8 membered heteroalkyl, optionally substituted C₅₋₈ cycloalkyl, optionally substituted 5-8 membered heterocycloalkyl, optionally substituted C₅₋₁₀ cycloalkylalkyl, optionally substituted 5-8 membered heterocycloalkyl-C₁₋₃-alkyl, optionally substituted phenyl, optionally substituted 5-8 membered heteroaryl, optionally substituted C₇₋₁₀ arylalkyl, and optionally substituted 5-8 membered heteroaryl-C₁₋₃-alkyl;
R⁵ is selected from the group consisting of C₁₋₆ alkyl, —NR⁶R⁷, —CH₂C(O)NH₂, and 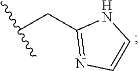 [SMILES: *Cc1ncc[nH]1]
and
R⁶ and R⁷ are independently selected from the group consisting of H, C₁₋₆ alkyl, and —CH₂C(O)NH₂.

23. The method of claim 13, wherein the β-lactamase inhibitor is a compound having the structure of any one of Formulas (X)-(XVII): 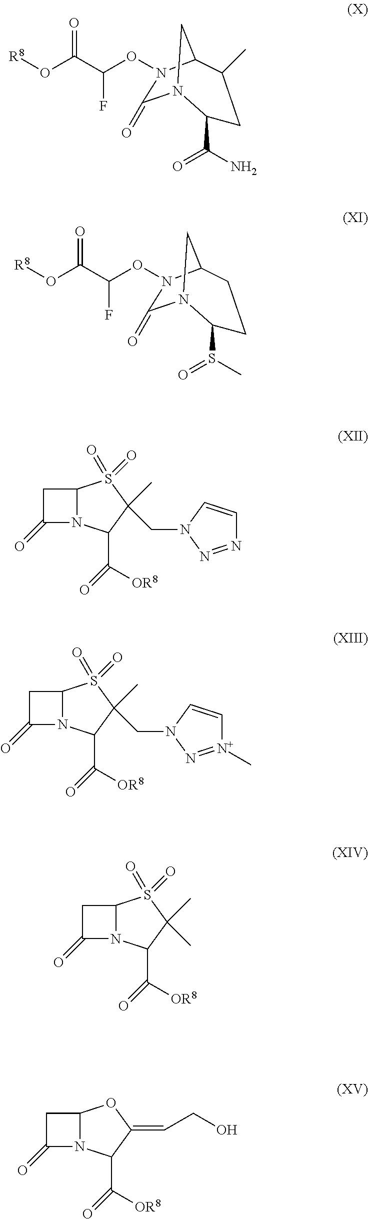 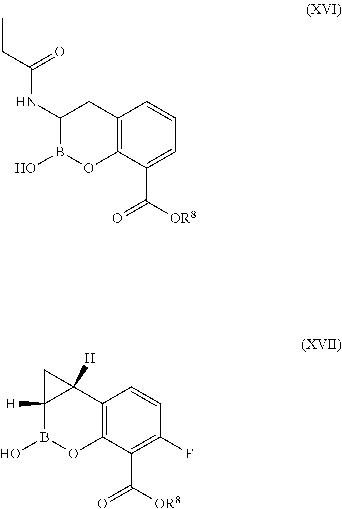
or pharmaceutically acceptable salts thereof, wherein:
R⁸ is selected from the group consisting of C₁₋₉ alkyl, —CR¹⁰R¹¹OC(O)C₁₋₉alkyl, —CR¹⁰R¹¹OC(O)C₃₋₇carbocyclyl, —CR¹⁰R¹¹OC(O)(3 to 7 membered heterocyclyl), —CR¹⁰R¹¹OC(O)C₂₋₈alkoxyalkyl, —CR¹⁰R¹¹OC(O)OC₁₋₉alkyl, —CR¹⁰R¹¹OC(O)OC₃₋₇carbocyclyl, —CR¹⁰R¹¹OC(O)O(3 to 7 membered heterocyclyl), —CR¹⁰R¹¹OC(O)OC₂₋₈alkoxyalkyl, —CR¹⁰R¹¹OC(O)C₆₋₁₀aryl, —CR¹⁰R¹¹OC(O)OC₆₋₁₀aryl, —CR¹⁰R¹¹C(O)NR¹³R¹⁴, —CR¹⁰R¹¹OC(O)O(CH₂)₁₋₃C(O)NR¹³R¹⁴, —CR¹⁰R¹¹OC(O)O(CH₂)₂₋₃OC(O)C₁₋₄ alkyl, —CR¹⁰R¹¹OC(O)O(CH₂)₁₋₃C(O)OC₁₋₄ alkyl, —CR¹⁰R¹¹OC(O)(CH₂)₁₋₃OC(O)C₁₋₄ alkyl, and 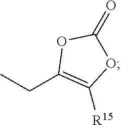
each R¹⁰ and R¹¹ is independently selected from the group consisting of H, optionally substituted C₁₋₄alkyl, optionally substituted C₃₋₇ carbocyclyl, optionally substituted 3-10 membered heterocyclyl, optionally substituted C₆₋₁₀aryl, and optionally substituted 5-10 membered heteroaryl;
each R¹³ and R¹⁴ is independently selected from the group consisting of H, optionally substituted C₁₋₆alkyl, optionally substituted C₃₋₇ carbocyclyl, optionally substituted 3-10 membered heterocyclyl, optionally substituted C₆₋₁₀aryl, and optionally substituted 5-10 membered heteroaryl; and
R¹⁵ is optionally substituted C₁₋₆ alkyl.

24. The method of claim 13, wherein the β-lactamase inhibitor is selected from the group consisting of: 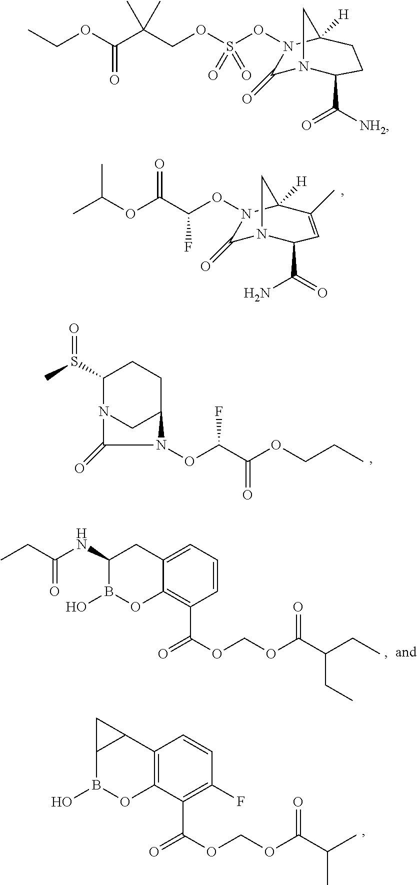
or pharmaceutically acceptable salts thereof.

25. The method of claim 13, wherein the amount of ceftibuten is administered once a day.

26. The method of claim 13, wherein the amount ceftibuten is administered twice a day.
